(19) United States
(12) Patent Application Publication
(10) Pub. No.: US 2013/0184567 A1
(43) Pub. Date: Jul. 18, 2013

(54) SYSTEMS AND METHODS OF POSITION AND MOVEMENT DETECTION FOR UROLOGICAL DIAGNOSIS AND TREATMENT

(71) Applicants: HUIKAI XIE, Gainesville, FL (US); JENSHAN LIN, Gainesville, FL (US); XIAOGANG YU, Gainesville, FL (US); HONGZHI SUN, Gainesville, FL (US)

(72) Inventors: HUIKAI XIE, Gainesville, FL (US); JENSHAN LIN, Gainesville, FL (US); XIAOGANG YU, Gainesville, FL (US); HONGZHI SUN, Gainesville, FL (US)

(73) Assignee: UNIVERSITY OF FLORIDA RESEARCH FOUNDATION, INCORPORATED, Gainesville, FL (US)

(21) Appl. No.: 13/550,025

(22) Filed: Jul. 16, 2012

Related U.S. Application Data
(60) Provisional application No. 61/510,178, filed on Jul. 21, 2011.

(51) Int. Cl. A61B 5/20; A61B 5/06

(52) U.S. Cl. CPC A61B 5/20; A61B 5/065
USPC 600/424; 600/595

(57) ABSTRACT

Systems and methods of detecting position and movement of the bladder and urethra are provided. In one embodiment, a system is provided including a flexible tube, or catheter, with one or more MEMS sensors and an RF antenna embedded thereon; and an RF receiver located external to the flexible tube. The sensors in the flexible tube can be connected to external electronics, which are in turn connected to a computing device. The MEMS sensors provide motion detection and the RF sensors provide position/distance detection. The MEMS sensors can include, but are not limited to, one or more accelerometers, gyroscopes, stress sensors, tilt sensors, and/or pressure sensors. In operation, the flexible tube is inserted into the bladder through the urethra to detect the location of the bladder neck and track the motion and/or shape of the urethra. The results of the tracking can be provided on a display.

### CROSS REFERENCE TO RELATED APPLICATION

The present application claims the benefit of U.S. Provisional Application Ser. No. 61/510,178, filed Jul. 21, 2011, which is hereby incorporated by reference herein in its entirety, including any figures, tables, or drawings.

### BACKGROUND

Many women suffer from Stress Urinary Incontinence (SUI), also known as effort incontinence, which is due to the insufficient strength of pelvic floor muscles. SUI refers to the accidental leaks of urine when coughing, laughing, sneezing, exercising, or other movements that increase intra-abdominal pressure, and thus increase pressure on the bladder. When there is insufficient strength of the pelvic floor muscles, hypermobility of the bladder neck can occur. In addition, since the urethra is supported by the fascia of the pelvic floor, the urethra will move downward during times of increased abdominal pressure if the support is insufficient. This movement of the urethra can result in allowing urine to pass.

In women, physical changes resulting from pregnancy and childbirth, as well as menopause often contribute to SUI. For example, pregnancy and childbirth can result in tearing of the tissues that support the bladder and urethra. In addition, lowered estrogen levels, due to menopause, high-level athletics, or during the week before the menstrual period can also contribute to lower muscular pressure around the urethra, increasing chances of leakage.

In an attempt to correct this defect, various surgeries have been devised with the intent of repositioning the bladder and restoring the urethra to their proper places by either a vaginal or abdominal surgical approach.

However, the failure rate of these surgeries is as high as 60% due to the lack of an intuitive and definitive way of tracking the position of the urethra during the surgeries.

Currently, the surgeon pulls the bladder neck into an approximate position, usually through the vaginal wall. The position is approximate because the surgeon cannot actually see, and thus must assume, through experience, the correct position.

This assumption by the surgeon is later confirmed correct or incorrect through the passage of time and/or the willingness of the patient to complain about the SUI or the recurrence of the SUI, in which case, the patient would be subjected to yet another possibly unsuccessful surgery.

Each time a surgery is performed there is an increased amount of scar tissue. The general immobilization of the tissues will increase after subsequent surgeries, which further adversely affect the subsequent success/failure rates of these surgeries. There is a genuine need for the surgeon to be able to watch the bladder and its position in real time as the surgery progresses in order to avoid more surgeries and to correct the SUI during the initial procedure.

By watching the positioning in real time, the surgeon would be able to position the bladder neck and the urethra correctly and not have to guess at the proper placement. It would no longer be a blind procedure leaving the bladder too tight or, at times, too loose or subject to the happenstance of a correct positioning.

One of the most accurate tools currently available for diagnosing urinary incontinence is a cystourethrogram. The diagnosis of urinary incontinence using this method is based on difficult to interpret pressure variants, which may lead to a misdiagnosis of SUI where the cause is urge incontinence or a neurological defect. Often, presently available diagnostic methods test the patient in the dorsal lithotomy position. However, in this position, the SUI tends to not occur. Therefore, an ideal test would be performed under the same events that cause incontinence, such as coughing, running, jumping, etc., making the diagnosis of the etiology more accurate by monitoring the mobilization of the patient's pelvic floor during the event that causes the incontinence. A method of performing such a test is described in PCT/US2010/053712, filed Oct. 22, 2010, which is hereby incorporated by reference in its entirety.

### BRIEF SUMMARY

Systems and methods of tracking the location, deformation, stress, and motion of the bladder and urethra are provided. In accordance with one embodiment, microelectromechanical system (MEMS) sensors, such as accelerometers, gyroscopes, pressure sensors, and stress sensors are used in the subject systems and methods. In another embodiment, radio frequency (RF) sensors are used in the subject systems and methods. In yet a further embodiment, a combination of MEMS sensors and RF sensors are used in the subject systems and methods.

According to an embodiment, a system is provided including a flexible tube, or catheter (such as a Foley catheter), with one or more MEMS sensors and an RF antenna embedded thereon. The MEMS sensors provide motion detection and the RF sensors provide position detection. For the system, an RF receiver is located outside of the flexible tube. In operation, the flexible tube is inserted into the bladder through the urethra to detect the location of the bladder neck and track the motion and/or shape of the urethra. The MEMS sensors can include, but are not limited to, one or more accelerometers, gyroscopes, stress sensors, tilt sensors, or pressure sensors. The MEMS sensors can provide measurement of movements and orientations, whereas the RF position sensors provide an initial estimate of the bladder neck location and are used to calibrate the error of the MEMS motion sensors accumulated through slow drifting movements. In certain embodiments, only the MEMS components are used or only the RF components are used; however, the combination of MEMS and RF sensing allows the system to achieve a more comprehensive detection. In addition, in certain embodiments, multiple RF receivers are used to enhance detection accuracy or extract more information such as the position of bladder relative to other body parts.

In certain embodiments, multiple MEMS sensors are provided along the flexible tube to perform detection/tracking of multiple points. In one implementation, the shape of the urethra is profiled using multiple MEMS sensors embedded within a Foley catheter. Data from the multiple MEMS sensors is used to infer the location and shape of the urethra.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1A shows a Foley catheter with a plurality of MEMS accelerometers in accordance with an embodiment of the invention.

FIG. 1B shows acceleration components of gravity in three axes for a MEMS accelerometer of an embodiment of the invention.

FIG. 1C illustrates an estimation of the coordinates of the embedded MEMS devices in accordance with an embodiment of the invention.

FIG. 1D shows a representation of a MEMS-based motion-tracking catheter system according to an embodiment of the invention.

FIG. 1E is a photograph of a Foley with MEMS sensors installed inside in accordance with an embodiment of the invention.

FIG. 1F shows a picture of the measured Foley shape of the Foley with MEMS sensors of FIG. 1D displayed on a screen.

FIG. 2 shows a diagram of a system architecture of an RF positioning system in accordance with an embodiment of the invention.

FIG. 3A shows a system for real-time tracking of pelvic organ position and movement in accordance with an embodiment of the invention.

FIG. 3B is a photograph of a prototype of a system for real-time tracking of pelvic organ position and movement in accordance with an embodiment of the invention.

FIG. 4 shows a schematic cross-section at line A of FIG. 3 in accordance with an embodiment of the invention.

FIG. 5 is a photograph of the experimental prototype of an embodiment of the invention.

FIG. 6A shows a plot of measured voltage vs. distance between RX and TX antennas using the experimental prototype during a test conducted in the air.

FIG. 6B shows a plot of adjusted voltage vs. distance between RX and TX antennas using the experimental prototype during a test conducted in the air.

FIG. 7A shows a plot of measured voltage vs. distance between RX and TX antennas using the experimental prototype during a test conducted in a pig.

FIG. 7B shows a plot of adjusted voltage vs. distance between RX and TX antennas using the experimental prototype during a test conducted in a pig.

### DETAILED DISCLOSURE

Systems and methods of tracking the location, deformation, stress, and motion of the bladder and urethra are provided.

As used herein, the term “urethra” may be defined as the canal leading from the bladder, discharging the urine externally. See STEDMAN'S MEDICAL DICTIONARY, at page 2072 (28^{th} ed.). The term “urinary bladder” refers to a musculomembranous elastic bag serving as a storage place for the urine, filled via the ureters and drained via the urethra. Id. at page 226. The term “bladder neck” refers to the constricted portion (or ‘neck’) of the bladder and is defined herein as the smooth muscle of the neck of the bladder.

In the normal female, the bladder neck is above the pelvic floor and is supported predominantly by the pubovesical ligaments, the endopelvic fascia of the pelvic floor and levator ani. The levators contract with elevated intra-abdominal pressure, increasing urethral closure pressure to maintain continence. Often, this anatonomical arrangement alters after childbirth and with increasing age, such that the bladder neck lies beneath the pelvic floor, particularly when the intra-abdominal pressure rises, which may fail to maintain continence (stress incontinence as a result of urethral hypermobility).

In accordance with one implementation of the present invention, the subject tracking system is integrated with, for example, a Foley catheter. The position of the Foley catheter can be relayed by sensors of the subject system to a display. Below the firm tip of the Foley catheter, e.g., about ½ inch, a small section of the catheter device can be filled with normal saline solution in order to find the neck of the bladder. By operating with an image of where the bladder and urethra are in the patient relative to the pubic bone, the coccyx or the vagina in real time during the procedure, the surgeon would be able to pull the bladder and the urethra to a position considered normal under direct observation and not merely by guessing how tight or how loose to position the anatomy. Of course, the integrated catheter and movement and position detection system can be used in other applications where pelvic organs are studied, diagnosed, and/or corrected via surgery, such as described by PCT/US/2010/053712 (“Treatment of Female Stress Urinary Incontinence”), which is incorporated by reference in its entirety.

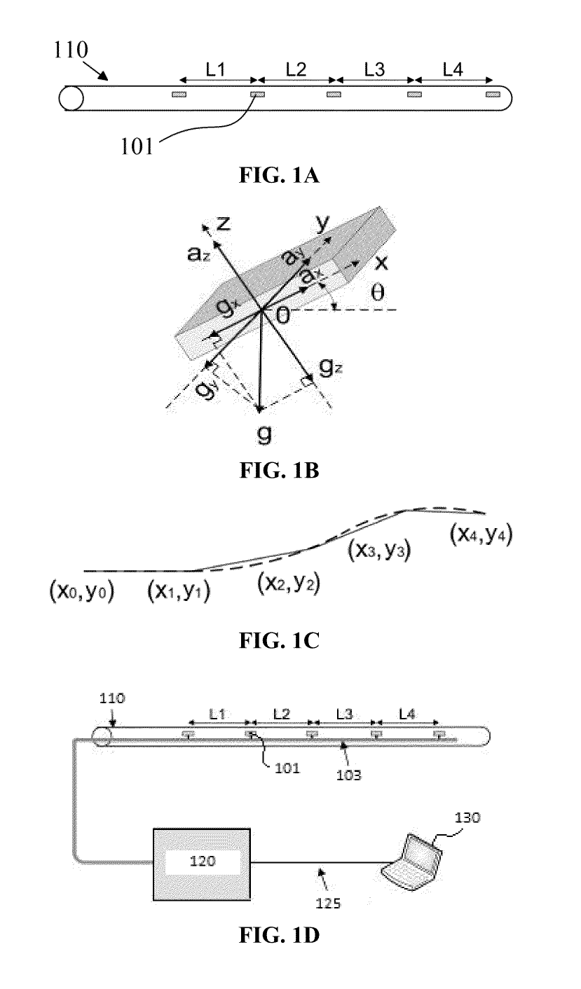

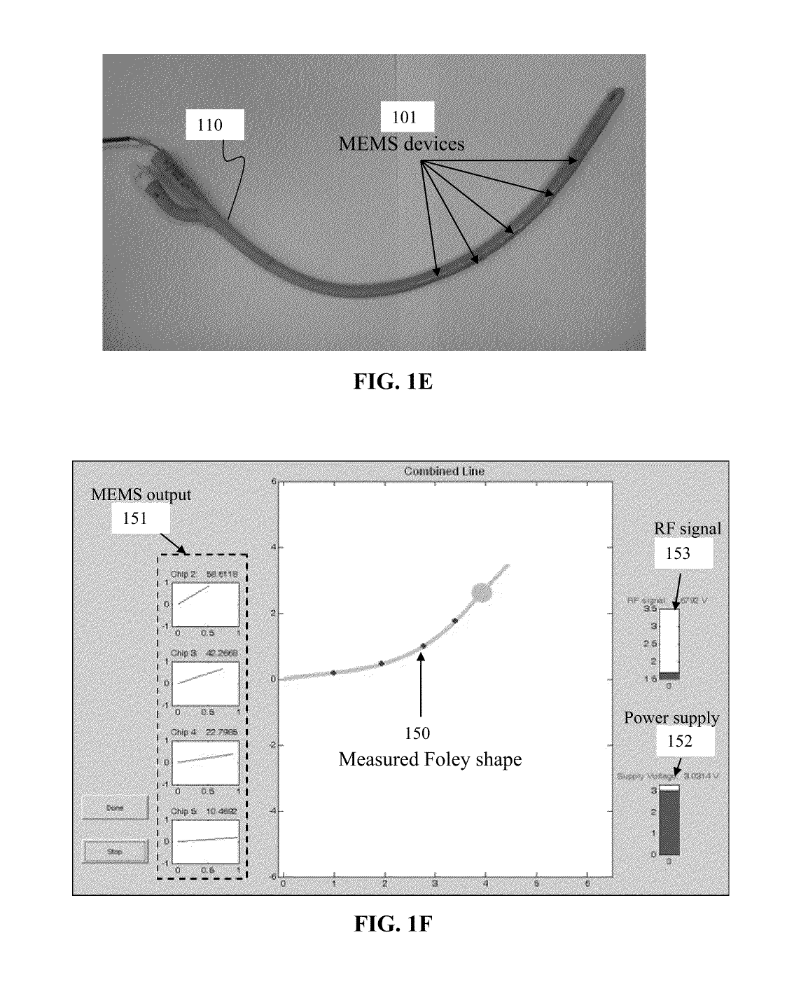

Referring to FIG. 1A, according to one embodiment of the invention, a plurality of MEMS devices 101 are mounted on different points inside a Foley catheter 110. The pitch of the MEMS devices 101 (length between adjacent devices) can be selected to provide sufficient data points while maintaining suitable flexibility of the catheter. The number of devices can be as few as three devices up to a number providing a pitch sufficient to maintain flexibility of the catheter. In one embodiment, 3-20 devices may be used. For example, 5-6 devices can be used. In certain embodiments, such as shown in FIG. 1A (and FIG. 4) the MEMS devices 101 can be arranged within the catheter 110 with a parallel orientation with respect to the length of the catheter. For example, as shown in FIG. 4, a MEMS chip arranged in parallel to the catheter has an edge visible in a cross-section of the catheter. In other embodiments, the MEMS devices 101 can be arranged within the catheter 110 perpendicular to the catheter such that a chip surface is visible in a cross-section of the catheter.

The MEMS device 101 can be provided in the form of a chip. Electrical connection of the MEMS chips to the outside may be provided by individual wires (for example, insulated copper wires). Alternatively, the MEMS chips can be mounted on a flexible PCB such that electrical connections are provided by the PCB

The MEMS devices 101 can be MEMS accelerometers such as the MEMS accelerometer shown in FIG. 1B. An angular resolution of less than one degree for the MEMS accelerometer is sufficient for the purposes of certain embodiments of the invention. Referring to FIG. 1B, a MEMS accelerometer can sense acceleration in one, two or three orthogonal axes (x, y, and z), and converts the acceleration signals to voltage as output. In the static case where there is no external acceleration, the device still sustains acceleration due to gravity that is equal to 1 g=9.8 m/s². If the MEMS device is tilted by an angle θ with respect to y axis, the acceleration at x axis and the value of θ can be expressed as

$${a}_{x} = g sin θ ; and$$

$$θ = - arcsin ( \frac{a}{\sqrt{{a}_{x}^{2} + {a}_{y}^{2} + {a}_{z}^{2}}} ) .$$

The tilting angle in the x-z plane is calculated based on the three axis outputs of the accelerometer. The value of aₓ does not change if an additional rotation happens along the direction of the gravity, providing one degree of freedom out of a three-dimensional (3-D) space not yet determined. In practical cases of women's SUI, the urethras' movement is limited by other organs within a 2-D space. Accordingly, tracking a urethra's motion can then be reduced to a 2-D problem. With multiple MEMS accelerometers mounted on different points along a Foley catheter with a certain pitch length, the tilting angles of those points on the catheter can be measured in real time, and the shape of the catheter can be described as the following equation:

y(x)=pₙxⁿ+pₙ₋₁xⁿ⁻¹+. . . +p₀

where x and y are the horizontal and the vertical coordinates of the points on the catheter, respectively (e.g., x is the coordinate along the catheter length direction and y is the deformation of the catheter in the vertical direction). Here, a polynomial equation is used to approximate the actual shape of the catheter, whose order n is equal to the number of MEMS accelerometers subtracted by one (e.g., where 4 MEMS devices are used, n=4−1=3).

Accordingly, by mounting the MEMS accelerometers at different points inside the Foley catheter, the tilting angle of each device can be calculated. As deformation occurs to the Foley catheter, the shape of the Foley is described by the above equation.

Referring to FIG. 1C, the coordinates of the MEMS devices can be calculated based on the piecewise line approximation given by:

$$( {x}_{0} , {y}_{0} ) = ( 0 , 0 )$$

$$( {x}_{n} , {y}_{n} ) = ( {\sum }_{k = 1}^{n} {L}_{k} cos {θ}_{k} , {\sum }_{k = 1}^{n} {L}_{k} sin {θ}_{k} ) ( n \geq 1 )$$

where Lₖ is the pitch length between adjacent MEMS devices and the devices' order 0, 1, . . . , n, are assigned as shown in FIG. 1C. Thus, the index matrix [pₙ . . . p₀] in the polynomial equation used to approximate the shape of the catheter can be calculated by substituting the piecewise line approximation into the polynomial equation used to approximate the shape of the catheter.

FIG. 1D shows a representation of a MEMS-based motion-tracking catheter system according to an embodiment of the invention. The MEMS-based motion-tracking catheter system shown in FIG. 1E includes a plurality of MEMS devices 101 in a flexible catheter 110. The output channels of the MEMS devices 101 are connected to a data acquisition (DAQ) card 120 (or other electronic circuitry including an analog to digital converter) through a bus 103 of insulated thin copper wires. The DAQ card 120 converts multiple-channel analog signals into a serial digital signal, and then feeds the digital signal to a computer 130 through a USB 125. Of course, in certain embodiments, the DAQ card 120 (or other circuitry) can communicate with the computer 130 by other connections and communication methods. The computer performs signal processing and visually displays the movement of the catheter 110.

FIGS. 1E and 1F respectively show a picture of a Foley 110 with MEMS devices 101 installed in accordance with an embodiment of the invention and a screen shot of the displayed Foley shape. The Foley catheter used in the prototype is a 26F (˜8.7 mm external diameter) Foley catheter. Five MEMS devices are built in to the catheter with a pitch length of about 1 cm. The MEMS accelerometers used in the example Foley catheter are LIS331AL from ST Microelectronics. This model is chosen for its small chip size (3 mm×3 mm×1 mm), which is suitable for the inner diameter of the Foley, as well as for its analog output signal, which provides good compatibility with the data acquisition (DAQ) card (USB 6210 from National Instruments) used in the prototype system.

Because it can be difficult to connect electrical wires directly on surface-mounted MEMS devices, one solution that is used in the subject prototype is to solder the MEMS devices on a flexible printed circuits board (PCB), where the internal electrical wirings are then implemented. The signals and the power supply are connected from the PCB to the DAQ card through thin insulated copper wires. The DAQ card converts the analog signals from the MEMS devices to a serial digital signal, and sends the data into a main computer through a standard USB port. The data collected by the main computer is processed immediately by a program in accordance with an embodiment of the invention running in MATLAB, which is a powerful tool for mathematical computation.

For a MEMS-only system, the program implements the equations described above with respect to the MEMS components via signal processing techniques including data calibration, filtering, performing calculations and, in some cases, addressing one or more of noise error, device misalignment error and dynamic error. For a combined MEMS/RF system, the program implements the equations described above with respect to the MEMS components and the equations described below with respect to the RF system components in order to correspond those values to a visual display of movement and/or position. The results are shown on the screen immediately so the shape of the Foley can be monitored in real time during the operation either for diagnosis or surgeries. Important data can also be stored on a hard disk for later reference. For the ease of maintenance of the system, additional information can be provided on the screen to a user. For example, in addition to the Foley shape 150, the subject system can provide the angles of all the devices (e.g., MEMS output 151), the power supply voltage 152, and the acquired signal 153 from an RF system (described in more detail below).

The MEMS devices track the urethra movement relative to the earth, instead of relative to the human body. Therefore, to more clearly track the urethra movement under conditions where a patient moves (e.g., jumping, walking, and coughing), RF detection can be incorporated. RF detection measures the distance between a transmitter antenna and a receiver antenna, so the urethra movements relative to the body can be detected. Therefore, the movement of the urethra relative to the human body can be tracked when the patients are not in static status. To enable positioning of a receiver antenna when the patients are not in static status, certain embodiments of the invention provide a waist belt with one or more built-in receiver antennas for patients to wear during diagnosis.

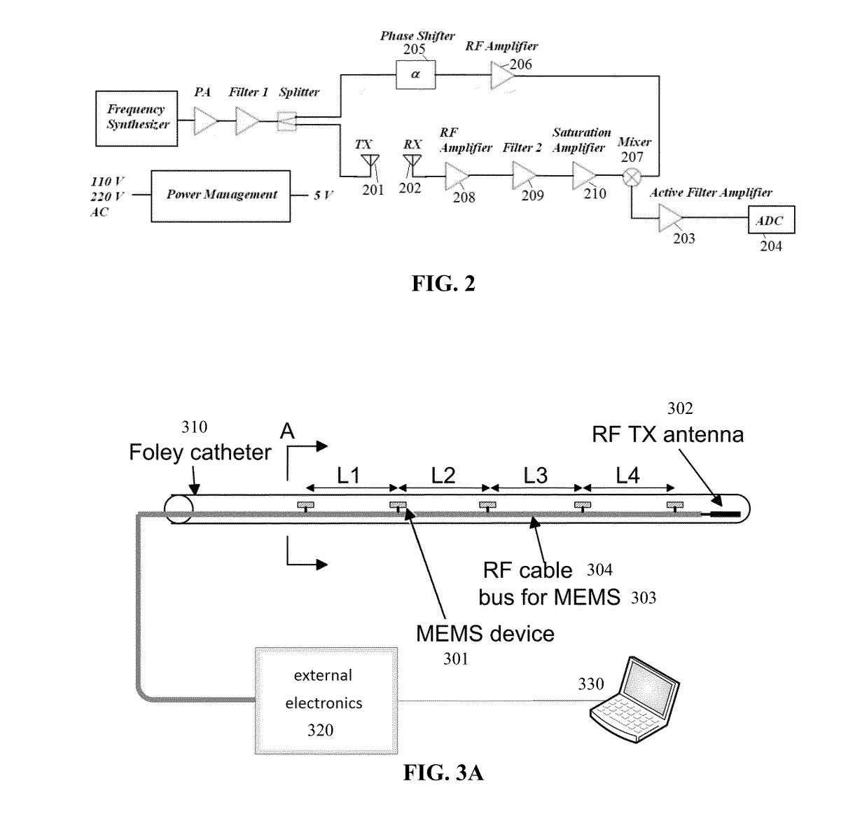

FIG. 2 shows a diagram of the RF architecture in accordance with an embodiment of the invention. In accordance with certain embodiments of the invention, phase-quadrature synchronous demodulation is used to detect phase shifting, and thereby determine movement of the transmitting portion with respect to the receiving portion.

Referring to FIG. 2, an RF signal is transmitted from a transmitting (TX) antenna 201, which can be provided in the Foley catheter, to a receiving (RX) antenna 202, which is provided external to the Foley catheter. The electromagnetic coupling occurs between the TX and RX antenna, where a displacement of the TX antenna 201 changes the phase shift of the signal received by the RX antenna 202. By comparing the RX signal and a reference local oscillator (LO) signal using a mixer/phase detector, the phase shift can be extracted because the phase shift is directly proportional to the displacement when the RX and LO signals are in quadrature. The output of the mixer/phase detector is a small DC voltage proportional to the phase shift.

In one implementation, an active filter amplifier 203 is used to amplify the small DC signal to a level that can be accepted by an analog-to-digital converter (ADC) 204 of a computer or other data acquisition interface. The active filter amplifier also reduces or eliminates the high frequency harmonics introduced by the RF amplifiers and the mixer in the preceding stages. In certain embodiments, the ADC 204 may be a stand-alone device.

The transmitting (TX) signal E_{TX} can be described as E_{TX}=E cos(ωt+φ_{TX}), where ω is the frequency of the TX signal in the unit of rad/s, and φ_{TX} is the initial phase of the TX signal. In one implementation, the TX signal is generated using a frequency synthesizer and RF amplifier. After the coupling between TX and RX antenna, the receiver receives the signal E_{RX}, which can be described as

$${E}_{RX} = {( \frac{d}{{d}_{0}} )}^{- γ} E cos ( ω t + {ϕ}_{TX} + Δϕ ) .$$

where Δφ is the phase shift introduced by the coupling and γ is the coupling coefficient. If the distance between the TX and RX antennas is larger than the dimension of the TX antenna, then γ=1.

The local oscillator (LO) signal E_{LO} can be generated by shifting the phase of the TX signal by 90° (quadrature phase shift) and can be described as

$${E}_{LO} = {E}^{′} cos ( ω t + {ϕ}_{TX} - \frac{π}{2} ) .$$

As shown in FIG. 2, the LO signal is derived from the TX signal with a quadrature phase shift. In order to provide the LO signal, according to one embodiment, a phase shifter 205 receives the TX signal and the phase shifted TX signal output by the phase shifter 205 is amplified using an RF amplifier 206. This LO signal is then provided to a mixer 207 for phase-quadrature synchronous demodulation of the RX signal passed through an RF front end including an RF amplifier 208 and filter 209.

After the phase-quadrature synchronous demodulation of the mixer, the baseband signal can be written as

$${E}_{BB} = c \cdot {( \frac{d}{{d}_{0}} )}^{- γ} \cdot ( cos ( ω t + {ϕ}_{TX} + Δ ϕ ) \cdot cos ( ω t + {ϕ}_{LO} ) ) ,$$

where c is a constant representing the gains of the amplifiers.

The active filter amplifier 203, such as an active low pass filter, retains the baseband signal and reduces or eliminates the high frequency signals generated by the RF amplifiers (206, 208) and mixer 207. Accordingly, the signal V_{M} fed into a data acquisition module (of a computer or other processing device) can be described as

$${V}_{M} = c \cdot {( \frac{d}{{d}_{0}} )}^{- γ} \cdot cos ( {ϕ}_{0} + Δϕ ) \approx c \cdot {( \frac{d}{{d}_{0}} )}^{- γ} \cdot Δϕ , \\ where {ϕ}_{0} = {ϕ}_{tx} - {ϕ}_{LO} = \frac{π}{2} .$$

Therefore, by setting the TX and LO signal at quadrature and setting the measuring distance larger than the dimension of the TX antenna, i.e. γ=1, the measured voltage is linearly proportional to the phase shift. Since the phase shift is linearly proportional to the measuring distance, measuring V_{M} can provide the distance between the TX and RX antennas.

For example, the distance d between the TX and RX antennas in the linear coupling region can be calculated as

$$d = \frac{{V}_{0}}{{V}_{M} - {Kd}_{0}} \cdot {d}_{0} ,$$

where V_{M} is the measured voltage from the data acquisition module, V₀ is the constant voltage when only intrinsic phase shift is present, K is the ratio of voltage change to the distance change, and d₀ is a unit distance for the system. For one experimental prototype, d₀=1 mm.

In the case of measuring distance smaller than the dimension of the TX and RX antenna, a saturation amplifier 210 is added to the RF front-end of the receiver as shown in FIG. 2. The saturation amplifier amplifies the received RF signal to the power supply rail. After the saturation amplifier 210, the RF signal amplitude is no longer dependent on the antenna coupling and is a constant. The RF signal phase is still related to the phase shift introduced by the displacement of the TX antenna. The RF signal after saturation amplifier can be described as

E_{RX,saturated}=Eᵣₐᵢₗcos(ωt+φₜₓ+Δ_{φ}).

The baseband signal with saturation amplifier can be described as

E_{BB,saturated}=c·cos(ωt+φₜₓ+Δ_{φ})·cos(ωt+φ_{LO}).

The measured voltage from the data acquisition module is described as

V_{M,saturated}=c·cos(φ₀+Δ_{φ})≈c·Δ_{φ}.

By introducing the saturation amplifier, the coupling effect on RF signal amplitude is eliminated and the measured voltage is linearly proportional to Δ_{φ}, the phase shift introduced by the coupling. Measuring V_{M} can provide the distance between the TX and RX antennas.

According to an embodiment, a system is provided including a flexible tube, or catheter, with one or more MEMS sensors and an RF antenna embedded thereon. The MEMS sensors provide motion detection and the RF sensors provide position detection. The MEMS sensors can also provide the angle and shape of the catheter (as the catheter bends), while the RF sensors can measure the changes in distance. For the system, an RF receiver is located outside of the flexible tube. In certain implementations, multiple RF receivers are used. In one embodiment, RF transceivers are located within the catheter and on patches (similar to EKG patches) placed on the pubic bone, hip bones, and coccyx. The four-RF-transceiver system provides a multi-antenna RF network that can be used for detecting stress-induced bladder neck movement while performing random movement cancelation and addressing clutter noise and null detection point issues.

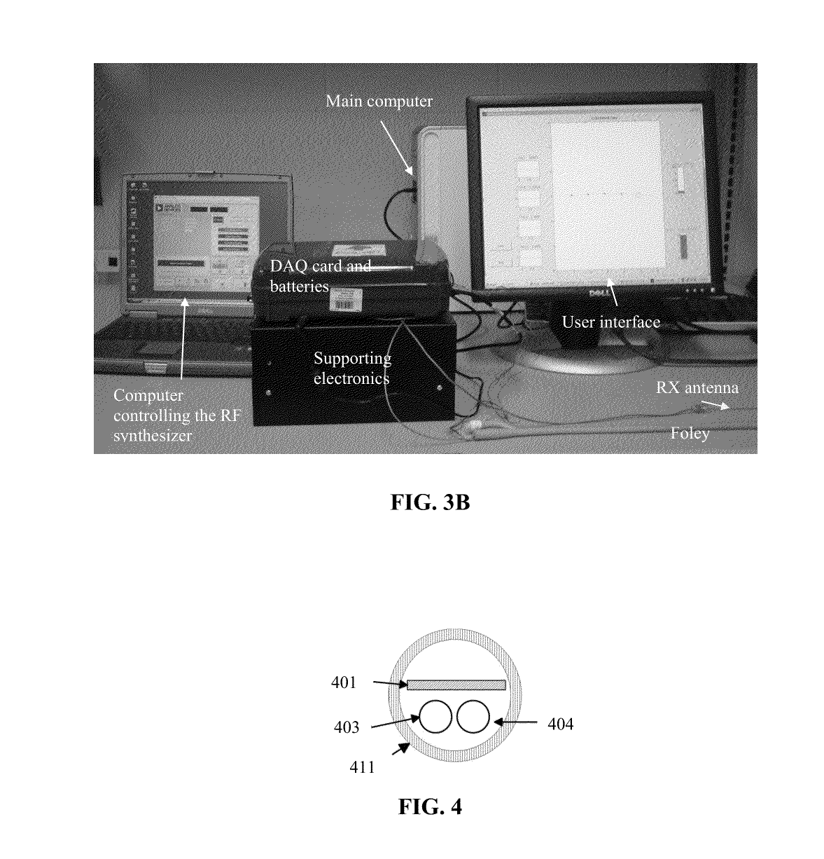

FIG. 3A shows a representation of the subject system integrated with a Foley catheter in accordance with an embodiment of the invention. FIG. 3B shows a photograph of such a system in accordance with a prototype example of an embodiment of the invention, in which four MEMS accelerometers are used.

Referring to FIG. 3A, multiple MEMS devices 301 and a single RF antenna 302 are embedded into a Foley catheter 310 in order to enable medical professionals to watch the urethra's shape and its movement relative to human body in a real-time environment. A MEMS bus 303 and RF cable 304 are connected to external electronics 320 and power supplies that may be needed but not provided within the Foley catheter 310.

As shown in FIG. 4, the MEMS devices 401, their signal bus 403 (e.g., for power and signal outputs), and an RF cable 404 for connection to the transmitting RF antenna (not shown) fit within the Foley catheter and can be embedded on the Foley wall 411.

Returning again to FIG. 3A, the external electronics 320 include the corresponding receiving RF antenna(s) and circuitry and can be connected to a computer 330. The computer 330 can be any suitable computing or processing device operably connected to a display. For example, the computer 330 may be, but is not limited to, a mobile device, desktop computer, or tablet. The external electronics 320 may include any standalone processing circuits or components that utilize the signals from the system, and which may provide additional processing or features before sending the data to the computer. Power management circuits can also be included as part of the external electronics 320.

The computer (or other device having a display screen) can provide a visual display of the shape and/or position of the urethra and/or bladder.

In certain cases, the external electronics and computer are combined into a single computer/processing system in which the functionality of both is provided. The single computer/processing system can be implemented as hardware including one or more computer processing units (CPUs), memory, mass storage (e.g., hard drive), and I/O devices (e.g., network interface, user input devices, data acquisition interface). Elements of the computer system hardware can communicate with each other via a bus (and in some cases a variety of cabling types). The computer/processing system may also be implemented, in part, in a distributed-computing environment where tasks are performed by remote-processing devices that are linked through a communications network or other communication medium.

In various embodiments, a tracking and positioning system can include the sensors (MEMS and/or RF) and a computer readable medium on which instructions are stored for execution by a processor. The instructions can be in the form of a computer program enabling certain signal processing steps as well as providing for displaying of graphs or images based on the signal processing steps and/or the equations described herein.

It should be appreciated by those skilled in the art that computer-readable media include removable and non-removable structures/devices that can be used for storage of information, such as computer-readable instructions, data structures, program modules, and other data used by a computing system/environment. A computer-readable medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); or other media now known or later developed that is capable of storing computer-readable information/data. Computer-readable media should not be construed or interpreted to include any propagating signals.

In operation, the flexible tube/catheter is inserted into the bladder through the urethra to detect the location of the bladder neck and track the motion and/or shape of the urethra. While MEMS accelerometers are described in detail herein, the MEMS devices are not limited thereto. For example, the MEMS sensors can include, but are not limited to, one or more accelerometers, gyroscopes, stress sensors, tilt sensors, or pressure sensors. The MEMS sensors can provide measurement of movements and orientations, whereas the RF position sensors provide an initial estimate of the bladder neck location and are used to calibrate the error of the MEMS motion sensors accumulated through slow drifting movements. In certain embodiments, only the MEMS components are used or only the RF components are used; however, the combination of MEMS and RF sensing allows the system to achieve a more accurate detection.

A greater understanding of the present invention and of its many advantages may be had from the following examples, given by way of illustration. The following examples are illustrative of some of the methods, applications, embodiments and variants of the present invention. They are, of course, not to be considered in any way limitative of the invention. Numerous changes and modifications can be made with respect to the invention.

### EXAMPLE

### Experimental Prototype

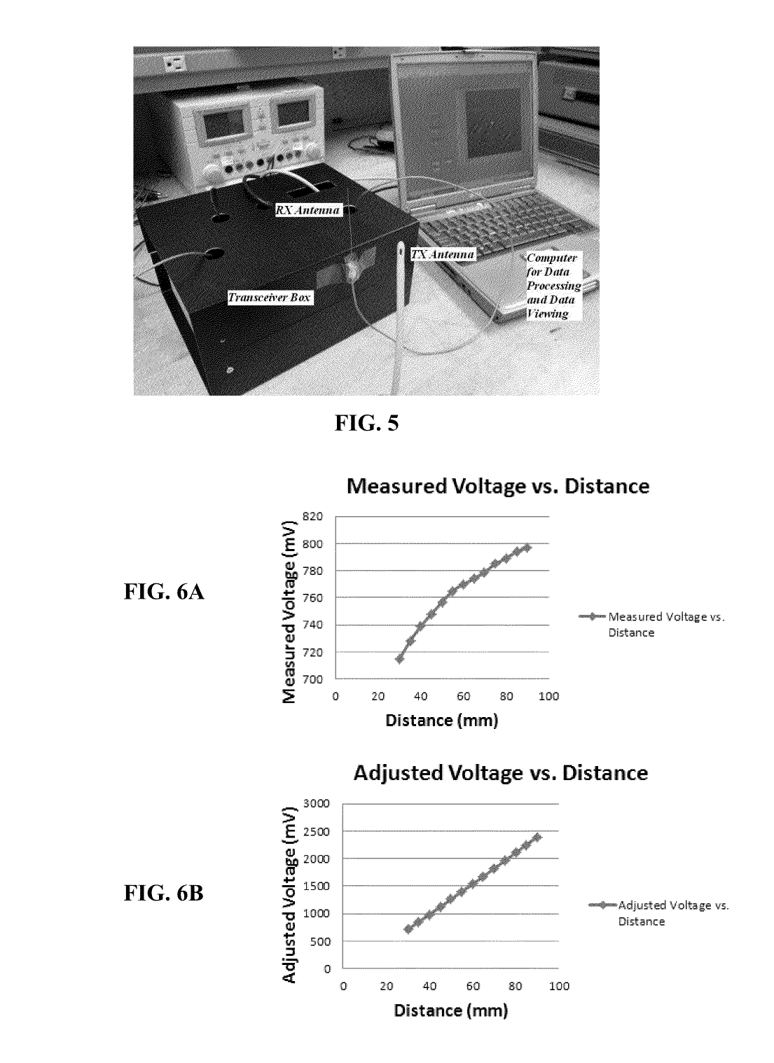

An experimental prototype of the RF system was created using the devices described in Table 1. FIG. 5 is a photograph of the prototype.

TABLE 1

| | | |
|---|---|---|
| Block | Manufacturer | Specifications |

Transmitter and Transmitting Antenna

| | | |
|---|---|---|
| Frequency | Analog | 65 to 400 MHz, −120 dBc/Hz typ @ 100 |
| Synthesizer | Devices | kHz phase noise, 0 dBm output power |
| Splitter | Minicircuits | 1-650 MHz, 36 dB isolation @ 90 MHz, 3.2 |
| | | dB total loss @ 90 MHz |
| Phase Shifter | Minicircuits | 80-210 MHz, 360 degree phase range, 10- |
| | | 12 dB return loss |
| RF Amplifier | RFMD | DC-8 GHz, 15.5 dB maximum gain, 13 |
| | | dBm P1 dB @1 GHz |
| RF Antenna | Made In | Copper, 2 cm-4 cm length, 1 mm diameter, |
| | House | 1 mW-10 mW output power |

Receiver and Receiving Antenna

| | | |
|---|---|---|
| RF Amplifier | RFMD | DC-8 GHz, 15.5 dB maximum gain, 13 |
| | | dBm P1 dB @1 GHz |
| Mixer | Minicircuits | 2 to 500 MHz, 5.3 dB conversion loss, 60 dB |
| | | LO to RF isolation @90 MHz, 3 dBm at LO |
| Baseband | TI | 5.1 MHz GBW, 10.5 V/us slew rate |
| Amplifier | | |

Data Acquisition

| | | |
|---|---|---|
| Data | National | 14-bit, 48 kS/s, USB powered |
| Acquisition | Instruments | |

Power Management and Enclosure Box

| | | |
|---|---|---|
| Power | Maxim IC | 2.7-11.5 V input voltage, 5 V output |
| | | voltage, 500 mA maximum load current |
| Enclosure | Hammond | Steel, 5″ height, 8″ width, 10″ depth |

### Example 1

### Prototype Air Test

The example prototype was tested in the air to test the ability of the RF design to detect position and movement. FIG. 6A shows measured voltage and FIG. 6B shows coupling-adjusted voltage when the RX and TX antennas are communicating through the air. Tests were performed with distances between the two antennas of between 30 nm to 90 nm. As can be seen from FIG. 6B, there is a linear relationship between the adjusted voltage and the distance.

### Example 2

### Prototype Pig Test

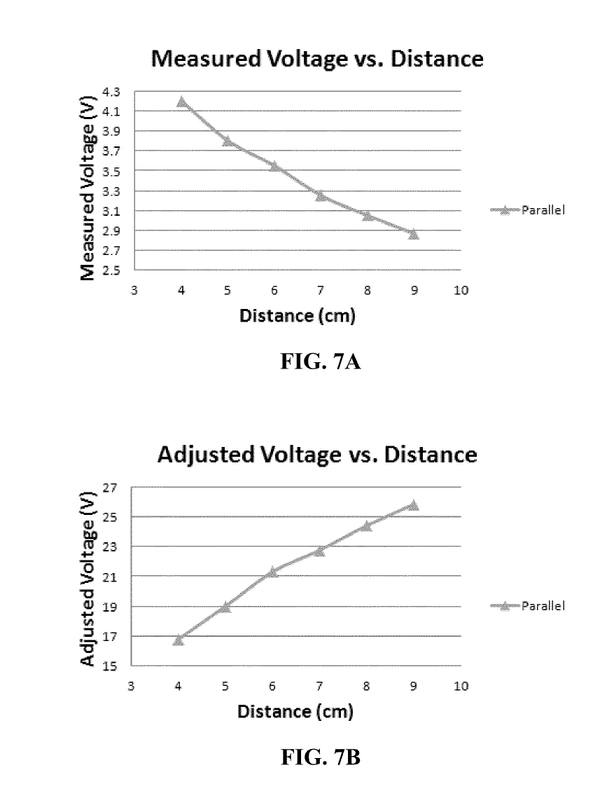

The example prototype was tested in a pig to test the ability of the RF design to detect position and movement through tissue. FIG. 7A shows measured voltage and FIG. 7B shows coupling-adjusted voltage when the RX and TX antennas are communicating through the pig. Tests were performed with distances between the two antennas of between 30 nm to 90 nm. As can be seen from FIG. 7B, there is a linear relationship between the adjusted voltage and the distance.

Any reference in this specification to “one embodiment,” “an embodiment,” “example embodiment,” etc., means that a particular feature, structure, or characteristic described in connection with the embodiment is included in at least one embodiment of the invention. The appearances of such phrases in various places in the specification are not necessarily all referring to the same embodiment. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

It should be understood that the examples and embodiments described herein are for illustrative purposes only and that various modifications or changes in light thereof will be suggested to persons skilled in the art and are to be included within the spirit and purview of this application.

We claim:

1. A system for urological diagnostics and treatment, the system comprising:
a flexible tube or catheter;
a plurality of MEMS sensors in the flexible tube or catheter to provide tracking of the flexible tube or catheter, wherein the MEMS sensors of the plurality of MEMS sensors are spaced apart from each other along the length of the flexible tube or catheter by a pitch providing sufficient data points while maintaining flexibility of the flexible tube or catheter; and
a wiring connected to the plurality of MEMS sensors for providing power to and signals from the plurality of MEMS sensors.

2. The system according to claim 1, wherein the plurality of MEMS sensors comprises at least one sensor type from the group consisting of an accelerometer, a gyroscope, a pressure sensor, a tilt sensor, and a stress sensor.

3. The system according to claim 1, wherein the pitch is about 1 cm.

4. The system according to claim 1, wherein the plurality of MEMS sensors comprises between 3 and 20 MEMS sensors.

5. The system according to claim 1, further comprising computer readable medium having instructions stored thereon that when executed by a processor cause a processor to process the signals from the plurality of MEMS sensors in order to track the flexible tube or catheter.

6. The system according to claim 5, wherein the coordinates of the MEMS sensors along the flexible tube or catheter are given as:
$$( {x}_{0} , {y}_{0} ) = ( 0 , 0 )$$
$$( {x}_{n} , {y}_{n} ) = ( {\sum }_{k = 1}^{n} {L}_{k} cos {θ}_{k} , {\sum }_{k = 1}^{n} {L}_{k} sin {θ}_{k} ) ( n \geq 1 )$$
where Lₖ is the pitch between adjacent MEMS sensors of the plurality of MEMS sensors, θₖ is the tilt angle of a MEMS sensor with respect to a y axis, and n is a total number of MEMS sensors of the plurality of sensors minus 1 such that the coordinates are provided in device order of 0 to n,
wherein the instructions cause the processor to determine a shape of the flexible tube or catheter according to
y(x)=pₙxⁿ+pₙ₋₁xⁿ⁻¹+. . . +p₀,
where pₙ, . . . , p₀ are calculated from the coordinates of the MEMS sensors, x is the coordinate along the catheter length direction and y is the deformation of the flexible tube or catheter in the vertical direction

7. The system according to claim 6, wherein the instructions further cause the processor to output an image of the determined shape of the flexible tube or catheter to a display.

8. The system according to claim 1, wherein the wiring comprises insulated copper wires connecting the MEMS sensors to an external from the flexible tube or catheter.

9. The system according to claim 1, wherein the wiring is provided by a flexible PCB on which the plurality of MEMS sensors are disposed.

10. The system according to claim 1, wherein a MEMS chip of the plurality of MEMS sensors is oriented parallel to a length direction of the flexible tube or catheter.

11. The system according to claim 1, wherein a MEMS chip of the plurality of MEMS sensors is oriented perpendicular to a length direction of the flexible tube or catheter.

12. The system according to claim 1, further comprising:
a transmitting (TX) antenna in the flexible tube or catheter, wherein the TX antenna transmits a TX signal;
a receiving (RX) antenna external to the flexible tube or catheter for being positioned at a location on a patient, wherein the RX antenna receives the TX signal and outputs an RX signal;
a phase shifter and amplifier providing a quadrature shifted reference local oscillator (LO) signal by phase shifting and amplifying the TX signal; and
a mixer mixing the RX signal and the LO signal to output an output signal including a DC voltage proportional to a phase shift between the LO signal and the RX signal, the output signal providing positioning information of the flexible tube or catheter.

13. The system according to claim 1, further comprising one or more accelerometers and/or one or more gyroscopes external to the flexible tube or catheter for placement on a patient to determine body motion of the patient in which the flexible tube or catheter is inserted.

14. The positioning system according to claim 1, further comprising one or more accelerometers and/or one or more gyroscopes for implantation in a patient to determine body motion of the patient in which the flexible tube or catheter is inserted.

15. A system for urological diagnostics and treatment, the system comprising:
a flexible tube or catheter;
a transmitting (TX) antenna in the flexible tube or catheter, wherein the TX antenna transmits a TX signal;
a receiving (RX) antenna external to the flexible tube or catheter for being positioned at a location on a patient, wherein the RX antenna receives the TX signal and outputs an RX signal;
a phase shifter and amplifier providing a quadrature shifted reference local oscillator (LO) signal by phase shifting and amplifying the TX signal; and
a mixer mixing the RX signal and the LO signal to output an output signal including a DC voltage proportional to a phase shift between the LO signal and the RX signal, the output signal providing positioning information of the flexible tube or catheter.

16. The system according to claim 15, further comprising:
an active filter amplifier low pass filtering and amplifying the output signal of the mixer;
an analog-to-digital converter (ADC) receiving an output of the active filter amplifier and converting the output of the active filter amplifier to a digital signal; and
a data acquisition module receiving the digital signal from the ADC for performing analysis of the digital signal and determining a measured voltage.

17. The system according to claim 16, wherein a distance d between the TX antenna and the RX antenna in a linear coupling region is calculated via a processor of the data acquisition module as
$$d = \frac{{V}_{0}}{{V}_{M} - {Kd}_{0}} \cdot {d}_{0} ,$$
where V_{M} is the measured voltage from the data acquisition module, V₀ is a constant voltage when only intrinsic phase shift is present, K is the ratio of voltage change to the distance change, and d₀ is a unit distance.

18. The system according to claim 17, wherein the measured voltage V_{M} is given as
$${V}_{M} = c \cdot {( \frac{d}{{d}_{0}} )}^{- γ} \cdot cos ( {ϕ}_{0} + Δϕ ) \approx c \cdot {( \frac{d}{{d}_{0}} )}^{- γ} \cdot Δϕ$$
$${V}_{M} = c \cdot {( \frac{d}{{d}_{0}} )}^{- γ} \cdot cos ( {ϕ}_{0} + Δϕ ) \approx c \cdot {( \frac{d}{{d}_{0}} )}^{- γ} \cdot Δϕ ,$$
where c is a constant determined by each gain stage in the system, Δ_{φ}is a phase shift introduced by coupling between the TX antenna and the RX antenna,
$${ϕ}_{0} = {ϕ}_{TX} - {ϕ}_{LO} = \frac{π}{2}$$
$${ϕ}_{0} = {ϕ}_{tx} - {ϕ}_{LO} = \frac{π}{2} ,$$
φ_{LO} is a phase of the LO signal, φ_{TX} is the initial phase of the TX signal, and γ is a coupling coefficient.

19. The system according to claim 17, further comprising a saturation amplifier between the RX antenna and the mixer for amplifying the received RF signal to a power supply rail, wherein the measured voltage V_{M} is given as
V_{M}=c·cos(φ₀+Δ_{φ})≈c·Δ_{φ}, where Δ_{φ}is a phase shift introduced by coupling between the TX antenna and the RX antenna, and c is a constant determined by each gain stage in the system.

20. The system according to claim 15, further comprising;
a plurality of MEMS sensors in the flexible tube or catheter; and
a computer readable medium having instructions stored thereon that when executed by a processor cause the processor to determine a position and shape of the flexible tube or catheter using output signals of the plurality of MEMS sensors, position coordinate information of each of the plurality of MEMS sensors, and the output signal from the mixer.
